(19) United States
(12) Patent Application Publication
(10) Pub. No.: US 2025/0129081 A1
(43) Pub. Date: Apr. 24, 2025

(54) METHANOGEN INHIBITORS

(71) Applicants: David RENNISON, Wellington (NZ); PASTORAL GREENHOUSE GAS RESEARCH LIMITED, Wellington (NZ)

(72) Inventors: David RENNISON, Wellington (NZ); Ian Kenneth BODDY, Wellington (NZ)

(21) Appl. No.: 18/694,356

(22) PCT Filed: Sep. 23, 2022
(86) PCT No.: PCT/NZ2022/050124

§ 371 Date: Mar. 21, 2024

(30) Foreign Application Priority Data
Sep. 24, 2021 (NZ) 780622
Jun. 10, 2022 (NZ) 789272

(51) Int. Cl. C07D 487/04; A23K 20/137; A23K 50/10; A61K 31/4245; A61P 1/14

(52) U.S. Cl. CPC C07D 487/04; A23K 20/137; A23K 50/10; A61K 31/4245; A61P 1/14

(57) ABSTRACT

The present invention relates to a new class of methanogen inhibitors for ruminants, in particular new pyromellitic diimide derivatives for ruminants. The invention also extends to the use of such derivatives in ruminants to reduce methane production in the rumen and/or to enhance productivity in the ruminant.

### FIELD OF INVENTION

The present invention relates to a new class of methanogen inhibitors for ruminants, in particular new pyromellitic diimide derivatives for ruminants. The invention also extends to the use of such derivatives in ruminants to reduce methane production in the rumen and/or to enhance productivity in the ruminant.

### BACKGROUND OF INVENTION

Methane is produced as a natural consequence of digestion of feed by bacteria, fungi and protozoa in ruminant animals. This fermentation leads to the production of volatile fatty acids and peptides for the host. Fermentation also produces copious quantities of CO₂ and H₂ which are used by methanogenic archaea within the rumen to produce methane, which is ultimately released from the rumen, mostly through eructation.

The methane forming methanogens are members of the Archaea, and are quite different in a number of features to bacteria, fungi and protozoa. Methanogens have a number of unusual and archaeal-specific features, including cell wall structures, lipids, cofactors, and amino acid synthesis pathways, as well as their signature energy metabolism that is linked to methane production. They represent only about 1-4% of the rumen microbial community. It is known that, due to the unique metabolic pathways of methanogens and their low numbers, methanogen-specific inhibitors can be developed that do not adversely affect the fermentation of feed.

It has been recognised that the release of such methane is deleterious for two reasons. One is that methane is a greenhouse gas and the other is that the methane loss represents a loss of energy for the ruminant. It has been previously recognised that if one could inhibit or reduce the release of methane from ruminants that the impact of methane on the environment and atmosphere would be reduced and productivity gains might be achieved in the ruminants.

It is therefore an object of the present invention to overcome the above mentioned difficulties or to at least provide the public with a useful alternative.

### SUMMARY OF INVENTION

In one aspect the present disclosure provides a compound of Formula I

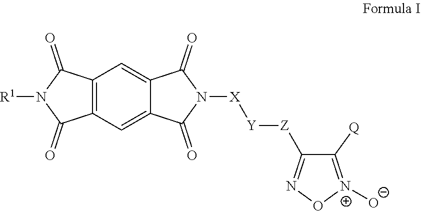

wherein R¹ may be selected from —H, -alkyl, -alkenyl, -alkynyl, -aryl, -benzyl or —CH₂ONO₂; or a group of Formula II

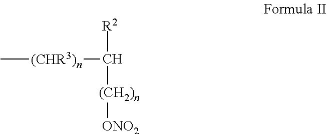

wherein each n may be independently selected from 0, 1, 2, 3, 4, 5 or 6 and when n is 0, 1, 2, 3, 4, 5, 6 any adjacent carbons are optionally unsaturated to form a double or triple bond,
wherein R² may be selected from —H, -alkyl, or -alkyl-ONO₂,
wherein each R³ may be independently selected from —H or -alkyl,
wherein X may be absent, —CH₂—, —CH(R⁴)—, —(CH₂)₂—, or —(CH₂)₃—,
wherein Y may be, —CH₂— or —CH(R⁵)—,
wherein Z may be absent, —O—, —S—, —NH— or —NR³—,
wherein R⁴ may be selected from —H or -alkyl,
wherein R⁵ may be independently selected from —H or -alkyl,
wherein Q may be —CN, —NO₂, —SOₘR⁶,
wherein R⁶ may be —H, -alkyl, -aryl or -benzyl,
wherein m is 0, 1 or 2,
or a salt thereof.

In one embodiment X is absent.

In one embodiment X is —CH(R³)— and R³ is -alkyl.

In one embodiment Y is —CH₂—.

In one embodiment Y is —CH(R³)— and R³ is -alkyl.

In one embodiment X is —CH₂— and Y is —CH₂—.

In one embodiment Z is absent.

In one embodiment Z is —O—.

In one embodiment R¹ may be —H.

In one embodiment R¹ may be -alkyl.

In one embodiment R¹ may be -alkenyl.

In one embodiment R¹ may be -alkynyl.

In one embodiment R¹ may be -aryl.

In one embodiment R¹ may be -benzyl.

In one embodiment R¹ may be —CH₂ONO₂.

In one embodiment n may be 0.

In one embodiment n may be 1.

In one embodiment n may be 2.

In one embodiment n may be 3.

In one embodiment n may be 4.

In one embodiment Q is —SOₘR⁶.

In one embodiment R⁶ is -aryl.

In one embodiment m is 2.

In one embodiment Q is —SO₂-phenyl.

In one embodiment the compound of Formula I is

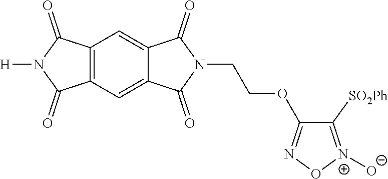

or a salt thereof.

In one embodiment the compound of Formula I is

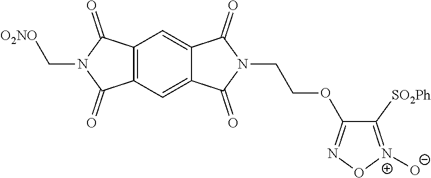

or a salt thereof.

In one embodiment the compound of Formula I is

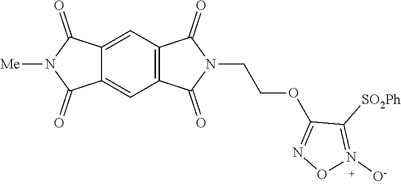

or a salt thereof.

In another aspect, the present invention provides the compound of Formula I as defined above for reducing the formation of methane from the digestive actions of ruminants and/or for improving ruminant performance.

In one embodiment a compound of Formula I as defined above is for reducing the formation of methane from the digestive actions of ruminants.

In one embodiment a compound of Formula I as defined above is for reducing the formation of methane from the digestive actions of ruminants by at least 10%.

In another embodiment of a compound of Formula I as defined above is for reducing the formation of methane from the digestive actions of ruminants by at least 15%.

In yet another embodiment the compound of Formula I as defined above is for reducing the formation of methane from the digestive actions of ruminants by at least 20%.

In one aspect the present invention further provides a method for reducing the production of methane emanating from a ruminant and/or for improving ruminant animal performance, comprising administering orally to the ruminant an effective amount of at least one compound of Formula I or a salt thereof to the ruminant. Oral administration is to be understood as a route involving drenching, addition to feed, water source or pasture; or manual administration of a bolus or a capsule.

In one embodiment the effective amount of at least one compound of Formula I or a salt thereof is administered at least once-daily to the ruminant.

In one embodiment the effective amount of at least one compound of Formula I or a salt thereof reduces the production of methane emanating from the ruminant by at least 10% per day.

In one embodiment the effective amount of at least one compound of Formula I or a salt thereof reduces the production of methane emanating from the ruminant by at least 15% per day.

In one embodiment the effective amount of at least one compound of Formula I or a salt thereof reduces the production of methane emanating from the ruminant by at least 20% per day.

In a further aspect of the present invention there is provided a composition for oral administration comprising at least one compound of Formula I or a salt thereof for reducing the production of methane emanating from a ruminant, and the composition further including at least one agriculturally and orally acceptable excipient.

In one embodiment the composition is adapted for use as a feed additive.

In one embodiment the composition is adapted for use as a water additive.

In another embodiment the composition is adapted for use as a ruminant lick.

In one embodiment the composition is adapted for use as an oral drench.

In another embodiment the composition is adapted for use as a rumen bolus or capsule.

In one embodiment the composition is adapted to reduce the production of methane emanating from the ruminant by at least 10% per day.

In one embodiment the composition is adapted to reduce the production of methane emanating from the ruminant by at least 15% per day.

In one embodiment the composition is adapted to reduce the production of methane emanating from the ruminant by at least 20% per day.

In one embodiment the excipient may include one or more minerals and/or one or more vitamins.

In one embodiment the excipient may include one or more vitamins selected from vitamin A, vitamin D3, vitamin E, and vitamin K, e.g. vitamin K3, vitamin B12, biotin and choline, vitamin B1, vitamin B2, vitamin B6, niacin, folic acid or the like.

In one embodiment the excipient may include one or more minerals selected from calcium, phosphorus, sodium, manganese, zinc, iron, copper, chlorine, sulphur, magnesium, iodine, selenium, and cobalt or the like.

In one embodiment the composition may further include sunflower oil, electrolytes such as ammonium chloride, calcium carbonates, starch, proteins or the like.

The compounds of the present invention have potential for use in a ruminant to reduce the formation of methane without affecting microbial fermentation in a way that would be detrimental to the ruminant.

### DETAILED DESCRIPTION OF INVENTION

### Definitions

The term “ruminant” as used herein is a mammal that is able to acquire nutrients from plant-based food by fermenting it in a specialized foregut (the rumen) prior to digestion, principally through microbial activity. Representative examples of ruminants and other foregut fermenters include cattle, goats, sheep, giraffes, bison, moose, elk, yaks, water buffalo, deer, camels, alpacas, llamas, and antelope.

The term “effective amount” as used herein refers to an amount of at least one compound of Formula I or a salt thereof that either reduces the production of methane emanating from the ruminant or improves ruminant performance.

The term “ruminant performance” as used herein refers to improving the productivity of the ruminant, such as increased weight gain, milk yield or quality, wool growth or quality, surviving offspring per parturition, or the like.

The term “halo” as used herein refers to a halogen atom selected from Cl, Br, or F.

The term “aliphatic ring” is a cyclic organic compound containing carbon atoms joined to form a non-aromatic ring. The aliphatic ring may be optionally substituted with alkyl, hydroxy, halo, —ONO₂ or the like.

The term “alkyl” as used herein refers to a straight or branched chain, saturated hydrocarbon having from 1 to 6 carbon atoms. Representative C₁-C₆ alkyl groups include, but are not limited to methyl, ethyl, propyl, isopropyl, butyl, iso-butyl, sec-butyl, tert butyl, pentyl (eg n-pentyl), hexyl and the like. The C₁-C₆ alkyl group may be further substituted with aryl.

The term “alkenyl” as used herein refers to a straight chain unsaturated hydrocarbon having from 2 to 6 carbon atoms including a single double bond. Representative C₂-C₆ alkenyl groups include, but are not limited to ethenyl (or vinyl), prop-1-enyl, but-1-enyl, pent-1-enyl, hex-1-enyl and the like.

The term “alkynyl” as used herein refers to a straight chain unsaturated hydrocarbon having from 2 to 6 carbon atoms including a single triple bond. Representative C₂-C₆ alkynyl groups include, but are not limited to ethynyl, prop-1-ynyl, but-1-ynyl, pent-1-ynyl, hex-1-ynyl and the like.

The term “aryl” as used herein refers to “aryl”, unless specifically limited, denotes a C₆₋₁₂ aryl group, suitably a C₆₋₁₀ aryl group, more suitably a C₆₋₈ aryl group. Aryl groups will contain at least one aromatic ring (e.g. one, two or three rings). Examples of a typical aryl group with one aromatic ring is phenyl. An example of a typical aryl group with two aromatic rings is naphthyl. The aryl group is optionally substituted with one or more —CN, -halo, —C(halo) 3, —NH₂, —NH-(alkyl), —N(alkyl) 2, alkyl, alkenyl, alkynyl, aryl or substituted aryl, the substituted aryl, may include a substituted phenyl (benzyl group), a substituted thiophenol, a tosyl group, the substituted aryl group being substituted with one or more —CN, -halo, —C(halo) 3, —NH₂, —NH-(alkyl), —N(alkyl) 2, alkyl, alkenyl, alkynyl,

All possible stereoisomers of the claimed compounds are included in the present disclosure. Where a compound described herein has at least one chiral center, it may accordingly exist as enantiomers. Where a compound possesses two or more chiral centers it may additionally exist as diastereomers. It is to be understood that all such isomers and mixtures thereof are encompassed within the scope of the present disclosure.

Some of the crystalline forms of the compounds may exist in more than one polymorphic form and as such all forms are intended to be included in the present disclosure. In addition, some of the compounds may form solvates with water (i.e., hydrates) or common organic solvents, and such solvates are also intended to be encompassed within the scope of this disclosure. The compounds, including their salts, can also be obtained in the form of their hydrates, or include other solvents used for their crystallization.

The present disclosure further includes within its scope prodrugs of the compounds described herein. In general, such prodrugs will be functional derivatives of the compounds which are readily convertible in vivo into the desired active compound. Thus, in these cases, the methods of treatment of the present invention, the term “administering” shall encompass the treatment of the various disorders described with prodrug versions of one or more of the claimed compounds, but which converts to the above specified compound in vivo after administration to the subject.

As used herein, the term “composition” is intended to encompass a product comprising a claimed compound(s) in a therapeutically effective amount, as well as any product which results, directly or indirectly, from combinations of the claimed compounds.

The term “or a salt thereof,” as used herein, is a salt of an acid or a basic nitrogen atom. Illustrative salts include, but are not limited, to sodium salt, potassium salt, lithium salt, calcium salt, ammonium salt, sulfate, citrate, acetate, oxalate, chloride, bromide, iodide, nitrate, bisulfate, phosphate, and the like.

### Methods of Making the Compounds of Formula I

### 3-(phenylsulfonyl)-4-(2-(1,3,5,7-tetraoxo-3,5,6,7-tetrahydropyrrolo[3,4-f]isoindol-2(1H)-yl)ethoxy)-1,2,5-oxadiazole 2-oxide (Compound 1)

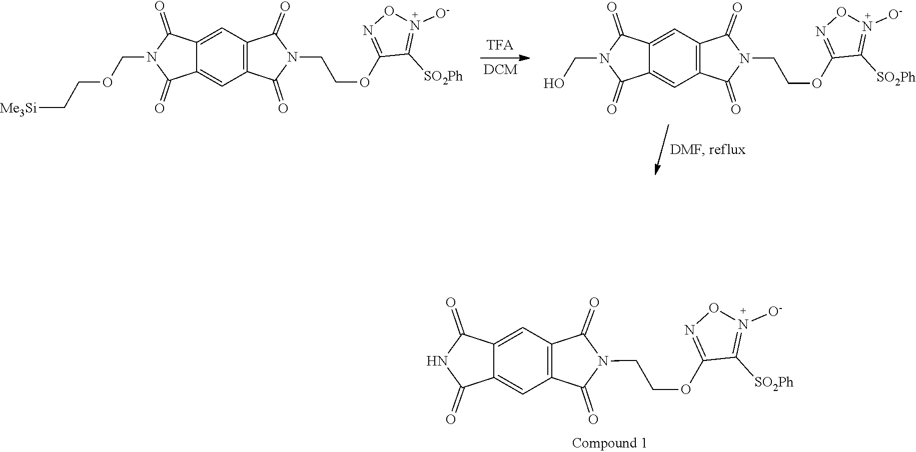

A solution of 3-(phenylsulfonyl)-4-(2-(1,3,5,7-tetraoxo-6-((2-(trimethylsilyl)ethoxy)methyl)-3,5,6,7-tetrahydropyrrolo[3,4-f]isoindol-2(1H)-yl)ethoxy)-1,2,5-oxadiazole 2-oxide (145 mg, 0.236 mmol) in dichloromethane/trifluoroacetic acid (5 mL, 4:1 v/v) was stirred at room temperature for 1.5 h, then concentrated in vacuo. The resulting residue was taken up in N,N-dimethylformamide (5 mL) and heated at reflux for 3 h. The reaction mixture was then quenched with water (20 mL) and extracted with ethyl acetate (3×20 mL). The combined organic layers were then washed with brine (3×20 mL), dried over MgSO₄, filtered and concentrated in vacuo. Purification by flash column chromatography (petroleum ether/EtOAc, 1:1) afforded Compound 1 as a colourless solid (61 mg, 0.126 mmol, 53%). ¹H NMR (500 MHz, d₆-DMSO) δ 4.12 (2H, t, J=5.4 Hz), 4.66 (2H, t, J=5.4 Hz), 7.65 (2H, dd, J=8.4 and 7.6 Hz)), 7.82 (1H, tt, J=7.6 and 1.2 Hz), 7.94 (2H, dd, J=8.4 and 1.2 Hz), 8.21 (2H, s), 11.87 (1H, s); ¹³C NMR (125 MHZ, d₆-DMSO) δ 36.5 (CH₂), 68.1 (CH₂), 110.5 (C), 117.4 (CH), 128.2 (CH), 129.9 (CH), 136.1 (CH), 136.8 (C), 137.2 (C), 138.1 (C), 158.7 (C), 166.1 (C), 167.7 (C).

### 4-(2-(6-((nitrooxy)methyl)-1,3,5,7-tetraoxo-3,5,6,7-tetrahydropyrrolo[3,4-f]isoindol-2(1H)-yl)ethoxy)-3-(phenylsulfonyl)-1,2,5-oxadiazole 2-oxide (Compound 2)

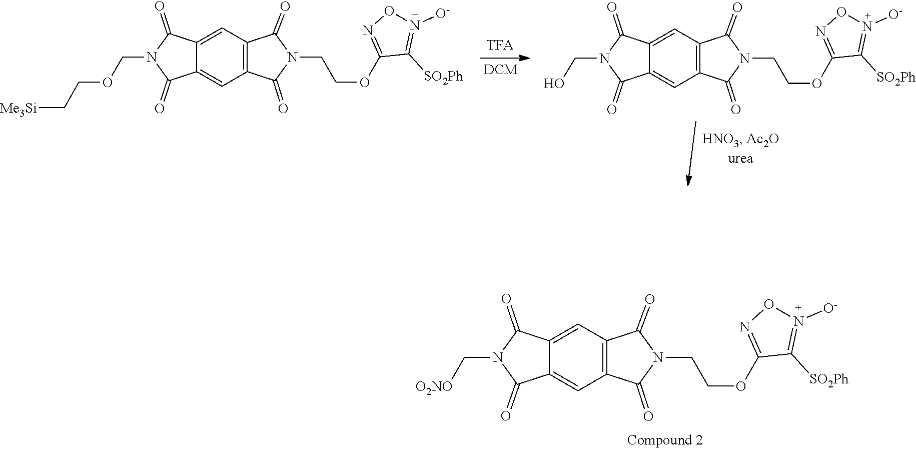

A solution of 3-(phenylsulfonyl)-4-(2-(1,3,5,7-tetraoxo-6-((2-(trimethylsilyl)ethoxy)methyl)-3,5,6,7-tetrahydropyrrolo[3,4-f]isoindol-2(1H)-yl)ethoxy)-1,2,5-oxadiazole 2-oxide (239 mg, 0.389 mmol) in dichloromethane/trifluoroacetic acid (10 mL, 4:1 v/v) was stirred at room temperature for 1.5 h, then concentrated in vacuo. The resulting residue was taken up in 1,2-dichloroethane (5 mL) and to this was added urea (1.2 mg, 0.019 mmol), acetic anhydride (0.18 mL, 1.94 mmol) and 70% nitric acid (0.06 mL, 0.972 mmol), and the mixture heated at reflux for 2 h. Following cooling to room temperature, the reaction mixture was first concentrated in vacuo, then diluted with ethyl acetate (20 mL) and washed with water (20 mL). The organic layer was then dried over anhydrous MgSO₄, filtered and concentrated in vacuo. Purification by flash column chromatography (petroleum ether/EtOAc, 6:4) afforded Compound 2 as a colourless solid (114 mg, 0.204 mmol, 52%). ¹H NMR (500 MHz, d₆-DMSO) δ 4.13 (2H, t, J=5.4 Hz), 4.66 (2H, t, J=5.4 Hz), 6.05 (2H, s), 7.66 (2H, dd, J=8.4 and 7.6 Hz), 7.82 (1H, t, J=7.6 Hz), 7.94 (2H, dd, J=8.4 and 1.2 Hz), 8.36 (2H, s); ¹³C NMR (125 MHz, d₆-DMSO) δ 36.6 (CH₂), 67.6 (CH₂), 68.1 (CH₂), 110.5 (C), 118.0 (CH), 128.2 (CH), 130.0 (CH), 136.1 (CH), 136.9 (C), 137.2 (C), 158.7 (C), 164.7 (C), 166.0 (C).

### 3-(Phenylsulfonyl)-4-(2-(1,3,5,7-tetraoxo-6-((2-(trimethylsilyl)ethoxy)methyl)-3,5,6,7-tetrahydropyrrolo[3,4-f]isoindol-2(1H)-yl)ethoxy)-1,2,5-oxadiazole 2-oxide

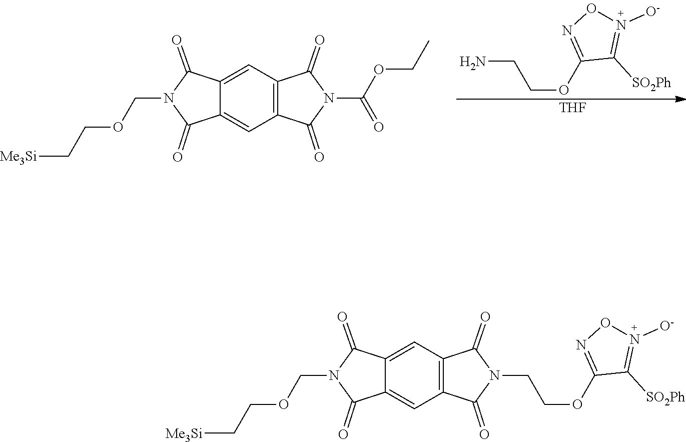

A solution of ethyl 1,3,5,7-tetraoxo-6-((2-(trimethylsilyl)ethoxy)methyl)-3,5,6,7-tetrahydropyrrolo[3,4-f]isoindole-2(1H)-carboxylate (100 mg, 0.239 mmol) and 4-(2-aminoethoxy)-3-(phenylsulfonyl)-1,2,5-oxadiazole 2-oxide (75 mg, 0.263 mmol) in tetrahydrofuran (10 mL) was stirred at room temperature for 1 h, then concentrated in vacuo. The resulting residue was taken up in ethyl acetate (20 mL) and washed with water (10 mL), followed by brine (10 mL). The organic layer was dried over anhydrous MgSO₄, filtered and concentrated in vacuo to afford the title compound as a yellow solid (145 mg, 0.236 mmol, 99%); which was used without further purification.

### Ethyl 1,3,5,7-tetraoxo-6-((2-(trimethylsilyl)ethoxy)methyl)-3,5,6,7-tetrahydropyrrolo[3,4-f]isoindole-2(1H)-carboxylate

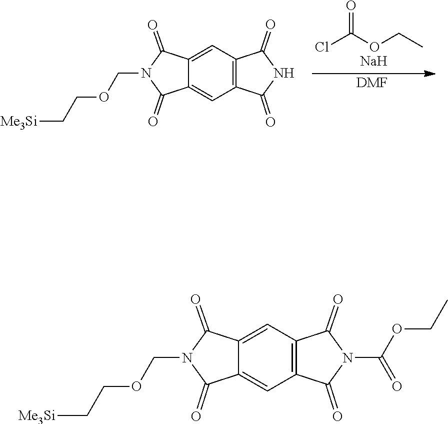

To a stirred solution of 2-((2-(trimethylsilyl)ethoxy)methyl) pyrrolo[3,4-f]isoindole-1,3,5,7(2H,6H)-tetraone (2.0 g, 5.77 mmol) in N,N-dimethylformamide (25 mL) was added sodium hydride (393 mg, 60% dispersion in mineral oil, 6.93 mmol), and the mixture stirred at room temperature for 30 min. Ethyl chloroformate (0.66 mL, 6.93 mmol) was then added dropwise, and the mixture stirred at room temperature for a further 1 h. The reaction mixture was then quenched with water (150 mL) and extracted with ethyl acetate (2× 60 mL). The combined organic layers were then dried over anhydrous MgSO₄, filtered and concentrated in vacuo to afford the title compound as a colourless solid (2.2 g, 5.26 mmol, 91%). ¹H NMR (400 MHZ, d₆-DMSO) δ −0.03 (9H, s), 0.88 (2H, dd, J=8.9 and 7.3 Hz), 1.34 (3H, t, J=7.1 Hz), 3.62 (2H, dd, J=8.9 and 7.3 Hz), 4.41 (2H, q, J=7.1 Hz), 5.01 (2H, s), 8.35 (2H, s); ¹³C NMR (100 MHz, d₆-DMSO) δ−1.4 (CH₃), 13.9 (CH₃), 17.3 (CH₂), 63.4 (CH₂), 66.3 (CH₂), 67.0 (CH₂), 118.7 (CH), 136.5 (C), 137.2 (C), 147.6 (C), 162.2 (C), 165.9 (C); ESI-MS: m/z calcd for C₁₉H₂₂N₂O₇Si: 418.1196; found [M+Na]⁺: 441.1073.

### 2-((2-(Trimethylsilyl)ethoxy)methyl) pyrrolo[3,4-f]isoindole-1,3,5,7(2H,6H)-tetraone

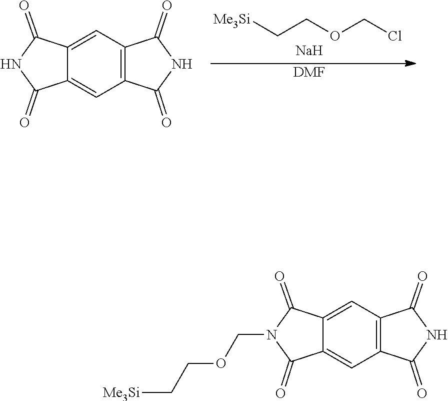

To a solution of pyromellitic diimide (2.00 g, 9.25 mmol) in N,N-dimethylformamide (200 mL) was added sodium hydride (0.24 g, 10.2 mmol, 60% dispersion in mineral oil), and the mixture stirred at room temperature for 30 min. 2-(Triemethylsilyl)ethoxymethyl chloride (1.80 mL, 10.2 mmol) was then added dropwise, and the mixture stirred at room temperature for a further 1 h. The reaction mixture was then quenched with water (200 mL) and extracted with ethyl acetate (2×100 mL). The combined organic layers were then washed with brine (3×200 mL), dried over anhydrous MgSO₄, filtered and concentrated in vacuo. Purification by flash column chromatography (petroleum ether/EtOAc, 3:1) afforded the title compound as a colourless solid (0.64 g, 1.85 mmol, 20%). ¹H NMR (400 MHZ, d₆-DMSO) δ−0.03 (9H, s), 0.87 (2H, dd, J=8.9 and 7.9 Hz), 3.61 (2H, dd, J=8.9 and 7.9 Hz), 5.01 (2H, s), 8.35 (2H, s), 11.86 (1H, s); ¹³C NMR (100 MHz, d₆-DMSO) δ−1.4 (CH₃), 17.3 (CH₂), 66.3 (CH₂), 66.9 (CH₂), 117.7 (CH), 136.6 (C), 138.2 (C), 166.2 (C), 167.5 (C); ESI-MS: m/z calcd for C₁₆H₁₈N₂O₅Si: 346.0985; found [M+Na]⁺: 369.0867.

### 4-(2-Aminoethoxy)-3-(phenylsulfonyl)-1,2,5-oxadiazole 2-oxide

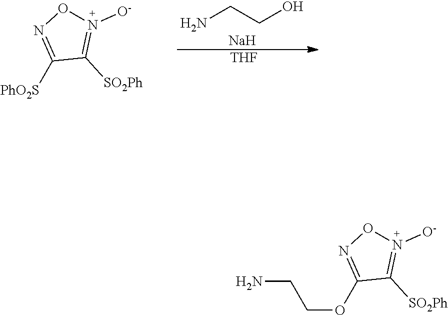

To a solution of 3,4-bis(phenylsulfonyl)-1,2,5-oxadiazole (0.60 g, 1.64 mmol), which is commercially available, in tetrahydrofuran (25 mL) at 0° C. was added ethanolamine (0.30 mL, 4.96 mmol), followed by sodium hydride (0.13 g, 3.25 mmol, 60% dispersion in mineral oil) portion-wise, and the mixture stirred at 0° C. for a further 3 h. The reaction mixture was then quenched with water (15 mL) and extracted with ethyl acetate (2×15 mL). The combined organic layers were then washed with brine (15 mL), dried over anhydrous Na₂SO₄, filtered and concentrated in vacuo to afford the title compound as a yellow oil (0.33 g, 1.16 mmol, 71%); which was used without further purification. ¹H NMR (400 MHZ, CDCl₃) δ 3.19 (2H, t, J=5.2 Hz), 4.46 (2H, t, J=5.2 Hz), 7.64-7.60 (2H, m), 7.78-7.74 (1H, m), 8.08-8.05 (2H, m).

### Pyromellitic Imide Anhydride

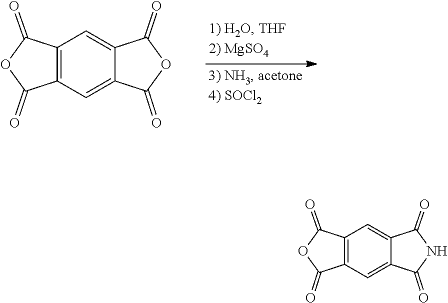 [SMILES: O=c1oc(=O)c2cc3c(=O)oc(=O)c3cc12 ; O=c1[nH]c(=O)c2cc3c(=O)oc(=O)c3cc12]

To a solution of pyromellitic dianhydride (20 g) in THF (200 mL) at room temperature was added water (11.4 mL), and the reaction stirred at room temperature for a further 15 min. After cooling to 0° C., anhydrous MgSO₄ (68.8 g) was added and the reaction stirred at 0° C. for a further 15 minutes, before allowing to warm to room temperature. After 30 minutes at room temperature the reaction mixture was filtered, and the collected solid washed with THF (2×100 mL). The filtrate was then cooled to 0° C. and saturated with anhydrous ammonia gas-rapidly forming a precipitate (at which point 200 mL of acetone was added to increase the efficiency of the stirring- and the mixture then re-saturated with further ammonia gas). After stirring at 0° C. for a further 20 minutes the insoluble material was then collected by filtration, washed with acetone and dried in vacuo to afford 2,4,5-tricarboxybenzamide as a colourless solid (24.6 g), which was used in the next step without further purification.

A solution of 2,4,5-tricarboxybenzamide (24.6 g) in thionyl chloride (200 mL) was heated at reflux for 2 hours. Upon cooling, hexane (200 mL) was added and the mixture stirred at room temperature for a further 30 minutes. The insoluble material was then collected by filtration, washed thoroughly with hexane, followed by ice-cold DMF:toluene (200 mL, 1:4 v/v) to remove the impurities (pyromellitic dianhydride and pyromellitic diimide). The resulting solid was then dissolved in acetone (400 mL) and any insoluble salts were filtered off. The collected filtrate was then concentrated in vacuo to afford compound pyromellitic imide anhydride as a light beige solid (16.4 g).

### 2-Methylpyrrolo[3,4-f]isoindole-1,3,5,7(2H,6H)-tetraone

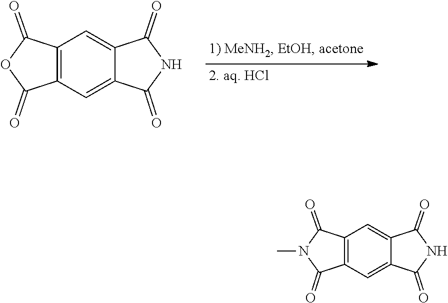 [SMILES: O=c1[nH]c(=O)c2cc3c(=O)oc(=O)c3cc12 ; Cn1c(=O)c2cc3c(=O)[nH]c(=O)c3cc2c1=O]

To a stirred solution of pyromellitic imide anhydride (5.00 g, 23.0 mmol) in acetone (150 mL) at 0° C. was added methylamine (7.20 mL, 33 wt. % in EtOH, 58.4 mmol) dropwise. The reaction mixture was stirred at room temperature for 30 min and concentrated in vacuo. Water (25 mL) was then added and the insoluble solids filtered off. The filtrate was then acidified to pH 1 with 2 M aq. hydrochloric acid solution and the mixture stirred at room temperature for 18 h. The precipitated solids were collected by filtration, washed with water (20 mL), then dried in vacuo to afford 2-methylpyrrolo[3,4-f]isoindole-1,3,5,7(2H,6H)-tetraone as a colourless solid (1.53 g, 6.65 mmol, 29%). ¹H NMR (400 MHZ, d₆-DMSO) δ 3.09 (3H, s), 8.13 (2H, s), 11.81 (1H, brs).

### Ethyl 6-methyl-1,3,5,7-tetraoxo-3,5,6,7-tetrahydropyrrolo[3,4-f]isoindole-2(1H)-carboxylate

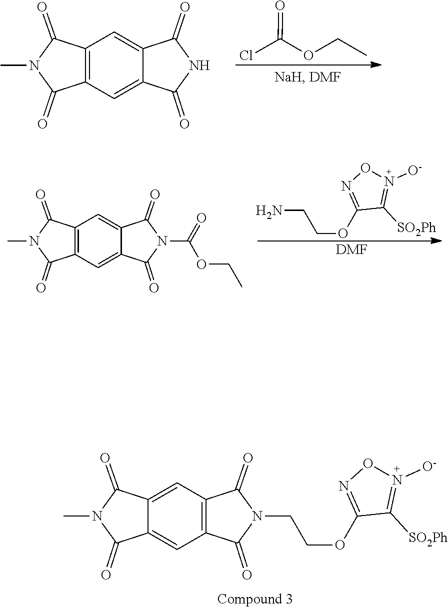

To a stirred solution of 2-methylpyrrolo[3,4-f]isoindole-1,3,5,7(2H,6H)-tetraone (100 mg, 0.434 mmol) in DMF (5 mL) was added sodium hydride (60% in mineral oil, 0.652 mmol, 1.5 eq.). The reaction mixture was stirred at room temperature for 30 min then ethyl chloroformate (62 μL, 0.652 mmol, 1.5 eq.) was added. After 24 h, the reaction mixture was quenched with water (20 mL) and then extracted with ethyl acetate (2× 20 mL). The combined organic layers were dried over anhydrous Na₂SO₄, filtered and concentrated in vacuo to yield a colourless solid (137 mg, a 1:1 mixture of product and starting material), which was used in the next step without further purification.

To the crude material (137 mg) in DMF (5 mL) was added 4-(2-aminoethoxy)-3-(phenylsulfonyl)-1,2,5-oxadiazole 2-oxide (91 mg, 0.317 mmol), and the mixture stirred at room temperature for 3 h. The reaction mixture was then concentrated in vacuo. Purification by flash column chromatography (petroleum ether/ethyl acetate, 1:1) afforded Compound 3 as a colourless solid (25 mg, 0.05 mmol, 11% over 2 steps). ¹H NMR (400 MHZ, CDCl₃) δ 3.25 (3H, s, CH₃), 4.26 (2H, t, J=5.2 Hz, CH₂), 4.72 (2H, t, J=5.2 Hz, CH₂), 7.60-7.64 (2H, m, ArH), 7.73-7.78 (1H, m, ArH), 8.01-8.04 (2H, m, ArH), 8.31 (2H, s, ArH); ¹³C NMR (100 MHz, CDCl₃) δ 24.5, 36.7, 67.4, 110.3, 118.4, 128.6, 129.7, 135.6, 137.0, 137.5, 158.4, 165.9, 166.1; HRMS (ESI/Q-TOF) m/z: [M+Na]⁺ calcd for C₂₁H₁₄N₄NaO₉S, 521.0374; found, 521.0363.

### In Vitro Studies

The compounds of the present invention were initially screened for activity using an in vitro fully automated incubation system for the measurement of total gas production and gas composition (methane and hydrogen). The system used is described in detail in the paper by Muetzel et al. (2014; Animal Feed Science and Technology 196, 1-11). The system in this specification is referred to as the rumen in vitro system or RIV. The RIV system provides a relatively simple tool for determining the rate and production of methane and also whether the rate and production of methane is inhibited or reduced by a particular compound or feed.

### Rumen In Vitro Assay Method.

Rumen in vitro assays use rumen fluid from donor animals (typically either sheep or cattle) which is typically combined with a buffer and then incubated in sealed fermentation vessels at 39° C. for either 24 or 48 h. These assays systems have been well-characterised and used by the scientific community over the last two decades (Rymer et al. 2005). The system that has been used here to characterise the effects of inhibitors on methane production and gas production, and the formation of H₂, was described by Muetzel et al. (2014). Rumen in vitro assay systems reflect what can occur in the rumen in vivo, but only short-term, due to their short incubation times (typically not exceeding 48 h) which is limited by its buffering capacity and the fact that it is a closed system.

Stock solutions of inhibitors were re-suspended in dimethylformamide (DMF) at concentrations 1000-fold higher than the highest used for the assays. Any further dilutions were also prepared using DMF. The total amount of inhibitor solution added to the rumen fluid-buffer mixture (60 mL) was 60 μl. Incubations used 60 ml of medium containing 12 ml of filtered rumen fluid and 48 ml of buffer in serum bottles for 24-48 h, essentially as described by Muetzel et al. (2014). Two fistulated cattle were used as donor animals for rumen fluid and treatments were incubated in duplicate bottles. Sets of duplicate incubation vials that contained ryegrass (with rumen fluid-buffer mixture), and ryegrass with 30 μM bromoethanesulfonate (BES) were also incubated as negative and positive controls, respectively. One experiment consists of 2 replicates of an inhibitor at any one concentration run at the same time, and a run can contain up to 32 bottle, including positive (30 UM BES) and negative (no inhibitor added) controls.

Compound 1, for example, was tested using the 60 mL rumen in vitro assay system at 30 μM, 10 μM, 2 μM and 1 μM final concentrations and compared to a positive control with 30 μM BES. At 30 μM and 2 μM, inhibition levels were approximately 87.1% and 23.3% at 18 h, respectively.

Compounds 2 and 3 of Formula I were also tested in the RIV using the same method and the RIV results are tabulated in Table 1. Compound 3 was very active in the RIV and only the lower concentrations of 2 μM and 1 μM were measured.

TABLE 1

| Compound | | RIV (all figures are % methane reduction) | | | |
|---|---|---|---|---|---|
| # | Structure | 30 μM | 10 μM | 2 μM | 1 μM |
| 1 | 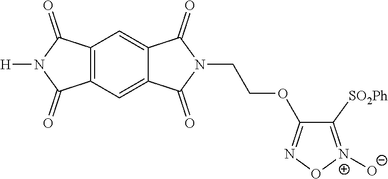 | 87.1 | 67.8 | 23.3 | 8.9 |
| 2 | 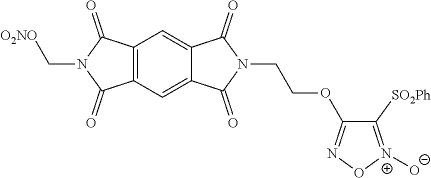 | 85.9 | 56.1 | 30.6 | 7.2 |
| 3 | 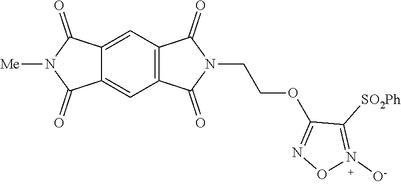 | — | — | 80.4 | 49.0 |

It is anticipated that the RIV results for the compounds of Formula I will be somewhat predictive of in vivo activity in reducing methane production in rumens.

The present invention and its embodiments have been described in detail. However, the scope of the present invention is not intended to be limited to the particular embodiments of the invention described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to the embodiments of the invention disclosed herein.

1. A compound of Formula I 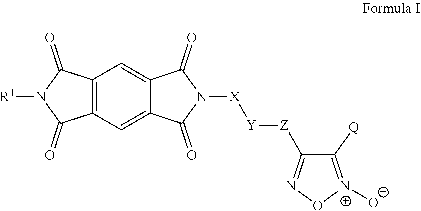
wherein R¹ may be selected from —H, -alkyl, -alkenyl, -alkynyl, -aryl, -benzyl or —CH₂ONO₂; or a group of Formula II 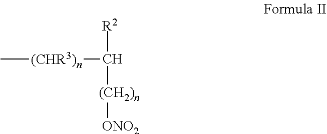
wherein each n may be independently selected from 0, 1, 2, 3, 4, 5 or 6 and when n is 0, 1, 2, 3, 4, 5, 6 any adjacent carbons are optionally unsaturated to form a double or triple bond,
wherein R² may be selected from —H, -alkyl, or -alkyl-ONO₂,
wherein each R³ may be independently selected from —H or -alkyl,
wherein X may be absent, —CH₂—, —CH(R⁴)—, —(CH₂)₂—, or —(CH₂)₃—,
wherein Y may be, —CH₂— or —CH(R⁵)—,
wherein Z may be absent, —O—, —S—, —NH— or —NR³—,
wherein R⁴ may be selected from —H or -alkyl,
wherein R⁵ may be independently selected from —H or -alkyl,
wherein Q may be —CN, —NO₂, —SOₘR⁶,
wherein R⁶ may be —H, -alkyl, -aryl or -benzyl, and
wherein m is 0, 1 or 2,
or a salt thereof.

2. The compound as claimed in claim 1, wherein X is absent.

3. The compound as claimed in claim 1, wherein X is —CH(R³)— and R³ is -alkyl.

4. The compound as claimed in any one of claims 1 to 3 wherein Y is —CH₂—.

5. The compound as claimed in any one of claims 1 to 3 wherein Y is —CH(R³)— and R³ is -alkyl.

6. The compound as claimed in claim 1 wherein X is —CH₂— and Y is —CH₂—.

7. The compound as claimed in any one of claims 1 to 6 wherein Z is absent.

8. The compound as claimed in any one of claims 1 to 6 wherein Z is —O—.

9. The compound as claimed in any one of claims 1 to 8, wherein R¹ is —H.

10. The compound as claimed in any one of claims 1 to 8, wherein R¹ is -alkyl.

11. The compound as claimed in any one of claims 1 to 8, wherein R¹ is -alkenyl.

12. The compound as claimed in any one of claims 1 to 8, wherein R¹ is -alkynyl.

13. The compound as claimed in any one of claims 1 to 8, wherein R¹ is -aryl.

14. The compound as claimed in claim 13, wherein R¹ is -benzyl.

15. The compound as claimed in any one of claims 1 to 8, wherein R¹ is —CH₂ONO₂.

16. The compound as claimed in any one of claims 1 to 8, wherein R¹ is a group of Formula II and each n is 0.

17. The compound as claimed in any one of claims 1 to 8, wherein R¹ is a group of Formula II and each n is 1.

18. The compound as claimed in any one of claims 1 to 8, wherein R¹ is a group of Formula II and each n is selected from 0, 1 or 2.

19. The compound as claimed in any one of claims 1 to 8, wherein R¹ is a group of Formula II and each n is selected from 0, 1 or 2, 3 or 4.

20. The compound as claimed in any one of claims 1 to 19, wherein Q may be —CN, —NO₂, or —SOₘR⁶.

21. The compound as claimed in claim 20, wherein Q is SOₘR⁶, m is 2 and R⁶ is -aryl.

22. The compound as claimed in claim 20 or claim 21, wherein Q is —SO₂-phenyl.

23. The compound as claimed in claim 1 wherein the compound of Formula I is 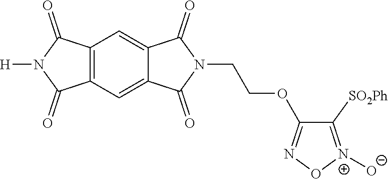
or a salt thereof.

24. The compound as claimed in claim 1 wherein the compound of Formula I is 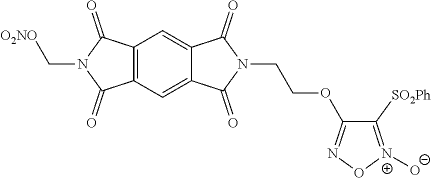
or a salt thereof.

25. The compound as claimed in claim 1 wherein the compound of Formula I is 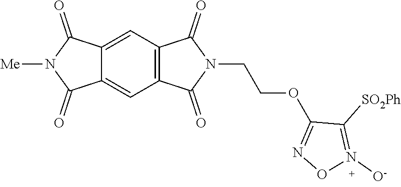
or a salt thereof.

26. The use of a compound of Formula I as claimed in any one of claims 1 to 25 for reducing the formation of methane from the digestive actions of ruminants and/or for improving ruminant performance.

27. The use of a compound of Formula I as claimed in any one of claims 1 to 25 for reducing the formation of methane from the digestive actions of ruminants.

28. The use of a compound of Formula I as claimed in any one of claims 1 to 25 for reducing the formation of methane from the digestive actions of ruminants by at least 10%.

29. The use of a compound of Formula I as claimed in any one of claims 1 to 25 for reducing the formation of methane from the digestive actions of ruminants by at least 15%.

30. The use of a compound of Formula I as claimed in any one of claims 1 to 25 for reducing the formation of methane from the digestive actions of ruminants by at least 20%.

31. A method for reducing the production of methane emanating from a ruminant and/or for improving ruminant animal performance, comprising administering orally to the ruminant an effective amount of at least one compound of Formula I or a salt thereof as claimed in any one of claims 1 to 25 to the ruminant.

32. The method as claimed in claim 31, wherein the effective amount of at least one compound of Formula I or a salt thereof is administered at least once-daily to the ruminant.

33. The method as claimed in claim 32, wherein the effective amount of at least one compound of Formula I or a salt thereof reduces the production of methane emanating from the ruminant by at least 10% per day.

34. The method as claimed in claim 33, wherein the effective amount of at least one compound of Formula I or a salt thereof reduces the production of methane emanating from the ruminant by at least 15% per day.

35. The method as claimed in claim 34, wherein the effective amount of at least one compound of Formula I or a salt thereof reduces the production of methane emanating from the ruminant by at least 20% per day.

36. A composition for oral administration comprising at least one compound of Formula I or a salt thereof as claimed in any one of claims 1 to 25 for reducing the production of methane emanating from a ruminant, and the composition further including at least one agriculturally and orally acceptable excipient.

37. The composition as claimed in claim 36 being adapted for use as a feed additive.

38. The composition as claimed in claim 36 or claim 37 being adapted for use as a water additive.

39. The composition as claimed in claim 36 or claim 37 being adapted for use as a ruminant lick.

40. The composition as claimed in claim 36 being adapted for use as an oral drench.

41. The composition as claimed in claim 36 being adapted for use as a rumen bolus or capsule.

42. The composition as claimed in any one of claims 36 to 41 being adapted to reduce the production of methane emanating from the ruminant by at least 10% per day.

43. The composition as claimed in claim 42 being adapted to reduce the production of methane emanating from the ruminant by at least 15% per day.

44. The composition as claimed in claim 41 being adapted to reduce the production of methane emanating from the ruminant by at least 20% per day.

45. The composition as claimed in any one of claims 36 to 44 wherein the excipient may include one or more minerals and/or one or more vitamins.

46. The composition as claimed in claim 45 wherein the excipient may include one or more vitamins selected from vitamin A, vitamin D3, vitamin E, and vitamin K, e.g. vitamin K3, vitamin B12, biotin and choline, vitamin B1, vitamin B2, vitamin B6, niacin, folic acid or the like.

47. The composition as claimed in claim 46 wherein the excipient may include one or more minerals selected from calcium, phosphorus, sodium, manganese, zinc, iron, copper, chlorine, sulphur, magnesium, iodine, selenium, and cobalt or the like.

48. The composition as claimed in any one of claims 36 to 47 further including sunflower oil, electrolytes such as ammonium chloride, calcium carbonates, starch, proteins or the like.
